(19) United States
(12) Patent Application Publication
(10) Pub. No.: US 2017/0306282 A1
(43) Pub. Date: Oct. 26, 2017

(54) CONTINUOUS FLOW SYSTEM

(71) Applicant: Apollonia Health Inc., Hamilton (CA)

(72) Inventors: Duane HEWITT, Hamilton (CA); Milos LEGNER, Mississauga (CA); Joon KIM, Hamilton (CA); Dennis Gerard CVITKOVITCH, Oakville (CA)

(21) Appl. No.: 15/159,128

(22) Filed: May 19, 2016

Related U.S. Application Data
(63) Continuation of application No. PCT/CA2016/000123, filed on Apr. 21, 2016.

(51) Int. Cl. C12M 1/00; C12M 3/00; G01N 33/483

(52) U.S. Cl. CPC C12M 23/20; G01N 33/4833; C12M 21/08; C12M 23/58

(57) ABSTRACT

A continuous flow system for passing fluid over a biofilm to simulate an oral environment is described. In one embodiment, the continuous flow system includes a plurality of channels fluidly connected by one or more channel connectors, an inflow conduit defining an inflow channel and an outflow conduit defining an outflow channel. The plurality of channels can receive the fluid via the inflow conduit from a reservoir positioned upstream of the flow cell housing and the outflow channel can receive the fluid from the plurality of channels.

### CROSS-REFERENCE TO RELATED APPLICATION

This application is a continuation of PCT/CA2016/000123, filed Apr. 21, 2016, the entire contents of which is hereby incorporated herein by express reference thereto.

### FIELD OF THE INVENTION

The present invention relates to a continuous flow system and method. In particular, the present invention relates to a continuous flow system and method for simulating an oral environment.

### BACKGROUND

An understanding of biotic and abiotic contributions to oral health and disease has been limited by the complexity of oral environments, as demonstrated by the plethora of interacting microorganisms inhabiting oral biofilms and a unique adaptation of each species to the composition of the biofilm and abiotic factors such as redox, pH and temperature, and the contents of nutrient and non-nutrient substances, including dissolved gases, in ambient fluids.

Static cultures of oral microorganisms, both single-species and mixed, are not expected to create an environment that emulates fluidic conditions of a normal human mouth. While flow systems for monitoring the growth of cells over time are known, none of those are suitable to sufficiently simulate an oral environment. Typically, the ex-vivo oral plaque samples are transferred to pre-sterilized well plate microfluidic (WPM) flow cells which fail to account for the complexity of the oral environment. A primary impediment associated with the WPM flow cells is that each cell consists of only a single flow channel constrained by a small diameter. The fixed channel design of known simulators is also a disadvantage because branching the flow path within the simulator is often required for experimental design. As a result, known oral environment simulators do not sufficiently reflect the composition of a biofilm in an actual oral environment, and are limited with respect to their ability to reproduce oral microbe growth patterns, rates of nutrient depletion, and responses of biofilms to changing environmental conditions.

Based on previously published data collected in vivo, (see e.g. Busher & van der Mei (2006), Clinical Microbiol. Rev. 19: 127; Nance et al. (2013), J. Antimicrob. Chemotherapy 68: 2550; Gupta (2014), “Viscometry for Liquids: Calibration of Viscometers”, Springer; Purcell (1977), Amer. J. Physics 45:3; Dawes et al., (1989), J. Dent. Res. 68: 1479), the ranges of fluidic parameters of a normal oral environment (during awake period) are known. The parameters are positively inter-correlated so that values higher than the normal range occur temporarily during meal consumption, values lower than the range occur only in certain regions of the mouth or during the night sleep. Specifically, three aspects of fluidic conditions have been suggested to influence the biofilm growth, namely dilution rate, shear stress and fluid velocity. Dilution rate (typical values of 11.1-19.0 h⁻¹) exerts selection pressure on cells in suspension (plankton) as well as provides exchange of fluids in the proximity of the biofilms. Shear stress (normal values 0.001-0.5 dyn cm⁻²) drives adhesion and release of cells to and from a wetted surface, such as the surface of a tooth. Fluid velocity (normal values 0.8-7.6 mm min⁻¹) in the proximity of the biofilm is a close correlate of the shear stress while in vivo, the existing methods allow for its more accurate estimate.

The reduced cross-sectional profile of a single flow channel in WPM flow cells leads to difficulties in modeling realistic dilution rate, shear stress and fluid velocity. For example, as the cross-sectional area of a channel is reduced, the effect of excessive dilution rates and shear stress is exacerbated by other physical phenomena, such as viscous forces dominating over inertial forces, and temperature and pressure-driven generation of micro-bubbles. The result is skewed biofilm growth profiles relative to an actual oral environment.

### SUMMARY

There is a growing awareness of the fundamental health impact of the complex microbial ecology that co-exists with the human body. However, systems and apparatuses are required to approximate conditions in subsets of microenvironments found in the mouth. Contrary to current setups, the system described herein can emulate the environment of the mouth by integrating a range of functions encountered in the living organism.

Effective simulation of oral conditions is critical to gain an understanding of interactions between abiotic and biotic factors which contribute to maintenance of oral health and/or onset of oral disease. A multitude of features can define a realistic modelling of an oral environment, including types and quantities of cells introduced into the flow system, types and availability of introduced nutrients, viscosity of ambient fluids, flow rate and resulting shear force, three-dimensional space availability for formation of a biofilm, degree of disturbance of the simulated environment during its operation, and abiotic factors.

A first aspect provided is a continuous flow system for passing fluid over a biofilm to simulate an oral environment, the continuous flow system comprising: a flow cell housing comprising: a base defining a longitudinal axis; and a plurality of channels defined by a plurality of channel walls supported by the base, the plurality of channels distributed adjacent to one another along the longitudinal axis of the base, each channel of the plurality of channels extending transverse to the longitudinal axis of the base, each channel of the plurality of channels having an inflow connection location for receiving the fluid into the channel and an outflow connection location for exporting the fluid from the channel; a plurality of removable channel connectors, each channel connector defining a connecting channel fluidly coupling a pair of the plurality of channels by connecting the outflow connection location of an upstream channel of the plurality of channels and the inflow connection location of a downstream channel of the plurality of channels; an upstream inflow adaptor fluidly connected to the flow cell housing for removably connecting to an inflow conduit defining an inflow channel; and a downstream outflow adaptor connected to the flow cell housing for removably connecting to an outflow conduit defining an outflow channel; wherein at least one of the plurality of channel walls is for supporting growth of the biofilm, the plurality of channels is for receiving the fluid via the inflow conduit from a reservoir positioned upstream of the flow cell housing, and the outflow channel is for receiving the fluid from the plurality of channels.

A further aspect is a method of passing fluid over a biofilm to simulate an oral environment within a flow cell having a plurality of channels defined by a plurality of channel walls supported by a base defining a longitudinal axis, the plurality of channels distributed adjacent to one another along the longitudinal axis of the base, each channel of the plurality of channels extending transverse to the longitudinal axis of the base, the method comprising: fluidly coupling an inflow channel defined by an inflow conduit to a reservoir containing fluid; fluidly coupling the inflow channel to a first channel of the plurality of channels; fluidly coupling the first channel of the plurality of channels to a second channel of the plurality of channels using a channel connector, the channel connector fluidly coupling the first channel to the second channel via a connecting channel defined by a wall of the channel connector; fluidly coupling an outflow channel defined by an outflow conduit to the second channel of the plurality of channels; and passing the fluid from the reservoir to the inflow channel such that the fluid flows from the inflow channel to the first channel, from the first channel to the second channel, and from the second channel to the outflow channel to promote growth of the biofilm.

A further aspect is a continuous flow system for passing fluid over a biofilm to simulate an oral environment, the continuous flow system comprising: a flow cell housing comprising: a base defining a longitudinal axis; and a plurality of channels defined by a plurality of channel walls supported by the base, the plurality of channels distributed adjacent to one another along the longitudinal axis of the base, each channel of the plurality of channels extending transverse to the longitudinal axis of the base, each channel of the plurality of channels having an inflow connection location for receiving the fluid into the channel and an outflow connection location for exporting the fluid from the channel; an upstream inflow adaptor connected to the flow cell housing for removably connecting to an inflow conduit defining an inflow channel for directing the fluid to the plurality of channels; a downstream outflow adaptor connected to the flow cell housing for removably connecting to an outflow conduit defining an outflow channel for receiving the fluid from the plurality of channels; and a removable reservoir fluidly connected to the plurality of channels via the inflow conduit for supplying the fluid to the plurality of channels; wherein at least one of the plurality of channel walls is for supporting growth of the biofilm, the plurality of channels receives the fluid from the reservoir via the inflow conduit, and the outflow channel receives the fluid from the plurality of channels.

A further aspect is a continuous flow system for passing fluid over a biofilm to simulate an oral environment, the continuous flow system comprising: a flow cell housing comprising: a base defining a longitudinal axis; and a plurality of channels defined by a plurality of channel walls supported by the base, the plurality of channels distributed adjacent to one another along the longitudinal axis of the base, each channel of the plurality of channels extending transverse to the longitudinal axis of the base, each channel of the plurality of channels having an inflow connection location for receiving the fluid into the channel and an outflow connection location for exporting the fluid from the channel; the fluid passed over the biofilm during a first stage of operation to produce a first stage shear stress at a first pre-determined shear stress range, a first stage fluid velocity at a first pre-determined fluid velocity range, and a first stage dilution rate at a first pre-determined dilution rate range; the fluid passed over the biofilm during a second stage of operation to produce a second stage shear stress at a second pre-determined shear stress range, a second stage fluid velocity at a second pre-determined fluid velocity range, and a second stage dilution rate at a second pre-determined dilution rate range, at least one of the second pre-determined shear stress range, the second pre-determined fluid velocity range and the second pre-determined dilution rate range being outside of the respective corresponding first pre-determined shear stress range, first pre-determined fluid velocity range, and first pre-determined dilution rate range.

Other advantages of the invention will become apparent to those of skill in the art upon reviewing the present specification.

### BRIEF DESCRIPTION OF THE DRAWINGS

Embodiments of the present invention will be described with reference to the accompanying drawings, wherein like reference numerals denote like parts, and in which:

FIG. 1 is a schematic top-view of flow cells comprising flow cell housing, connectors and inflow and outflow conduits of a continuous flow system;

FIG. 2 is a perspective view of the flow cells of a continuous flow system placed in a microscope stage insert;

FIG. 3A is a schematic cross-sectional view of aseptically replaceable fluid reservoirs of a continuous flow system;

FIG. 3B is a perspective view of an outflow reservoir of a continuous flow system;

FIG. 4A is a schematic cross-sectional view of a flow interrupter of a continuous flow system;

FIG. 4B is a perspective view of a flow interrupter situated underneath a flow cell of a continuous flow system;

FIG. 5 is a perspective view of a continuous flow oral simulator channel being seeded in a sterile environment using a disposable syringe;

FIG. 6 is a perspective view of a one-stream embodiment of a continuous flow oral simulator;

FIG. 7 is a schematic cross-sectional view of a channel fluidly coupled to a downstream channel via a channel connector;

FIG. 8 is a schematic cross-sectional view of two channels fluidly coupled via a channel connector; and

FIG. 9 is a perspective view of a side-flow attachment facilitating transfer of fluid into a flow cell from a syringe.

### DETAILED DESCRIPTION OF THE PREFERRED EMBODIMENTS

It will be appreciated that for simplicity and clarity of illustration, where considered appropriate, numerous specific details are set forth in order to provide a thorough understanding of the exemplary embodiments described herein. However, it will be understood by those of ordinary skill in the art that the embodiments described herein can be practiced without these specific details. In other instances, well-known methods, procedures and components have not been described in detail so as not to obscure the embodiments described herein. Furthermore, this description is not to be considered as limiting the scope of the embodiments described herein in any way, but rather as merely describing the possible implementations of various embodiments that can be varied as known by a person of ordinary skill in the art.

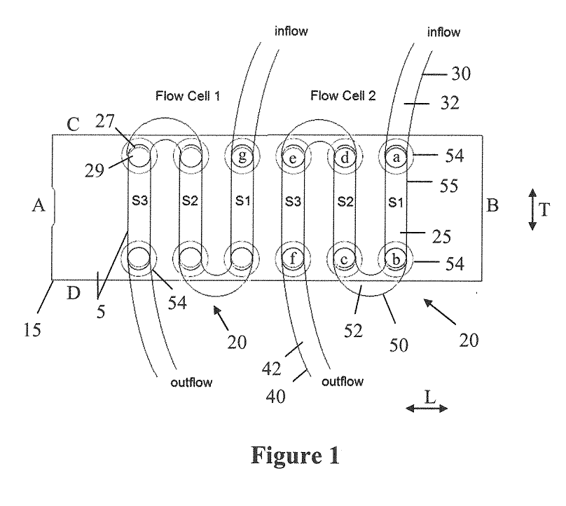

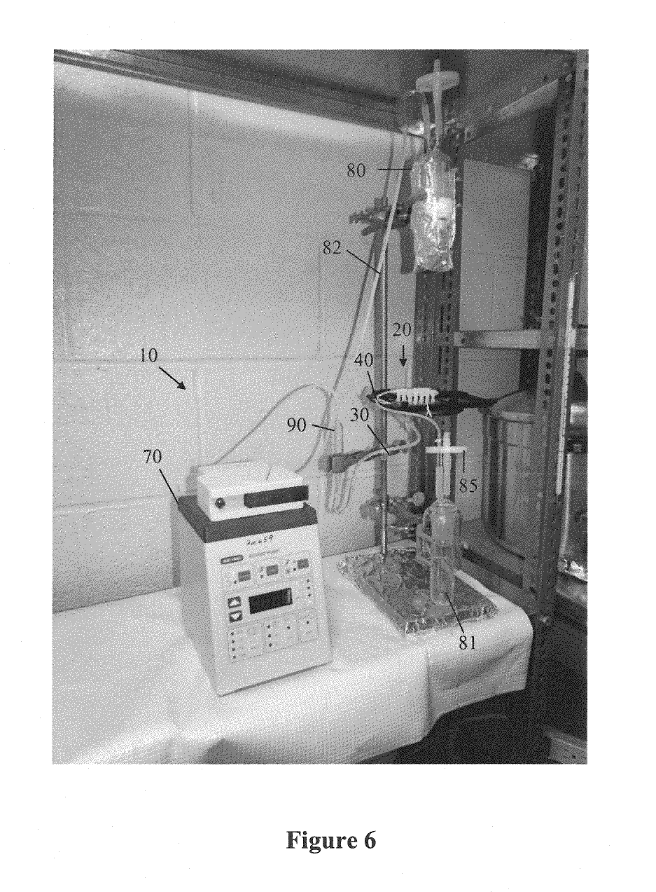

Referring to FIGS. 1 and 6, shown is a continuous flow system 10 having two flow cells 20. Each flow cell 20 comprises a flow cell housing 5 including a base 15 supporting a plurality of channel walls 55 defining a plurality of channels 25. The flow cell 20 can further include one or more channel connectors 50 each defining a connecting channel 52 for fluidly coupling two channels 25 via adaptors 54. Each flow cell 20 can have an inflow conduit 30 defining an inflow channel 32 for receiving fluid from a source reservoir 80 (e.g. via reservoir tube 82, hydraulic pump 70 and flow interrupter 90), and for delivering the fluid to a channel 25 of the flow cell 20 via adaptor 54. Each flow cell 20 can additionally include an outflow conduit 40 defining an outflow channel 42 for receiving fluid from a channel 25 via adaptor 54 and delivering the fluid to an outflow reservoir 81.

Herein the term “fluid” encompasses any liquid medium. For example, a fluid can be a liquid consisting essentially of a single liquid compound (e.g. deionized water) or more than one liquid compound (e.g. an aqueous ethanol solution). In other embodiments, a fluid can be a solution consisting of one or more liquid solvents and one or more solid solutes. For example, the fluid can be a nutrient broth for providing nutrients to cells. The nutrient broth can contain for example one or more amino acids, salts and sugars dissolved in water. In further embodiments, a fluid can consist of a liquid medium containing particles which are not dissolved (i.e. a suspension). An example of a suspension is a mixture of water and fluorescent beads. In certain embodiments, the fluid is a suspension containing cells. For example, a fluid can be a suspension comprising one or more species or sub-species of cells, or a dispersed sample of dental plaque, mixed in a nutrient broth solution. In one particular embodiment, a fluid comprises natural saliva. In another embodiment, a fluid comprises an artificial saliva composition. In particular embodiments, a fluid contains molecules for probing a biofilm established on the interior surface of a channel wall 55. For example, the fluid can contain molecular probes (e.g. labelled with fluorescent or radioactive moieties) capable of recognizing and binding to molecular targets on the surface of or within cells of the biofilm, or in the extracellular matrix surrounding the cells of the biofilm. In other embodiments, the fluid can contain one or more non-labelled compounds for altering the biofilm to observe a response of the cells in the biofilm to the one or more compounds. Non-limiting examples of compounds that can be contained in a fluid include proteins, amino acids, nucleic acids, sugars, polysaccharides, nucleosides, lipids, and drugs/pharmacological compounds.

Herein the term “cell” encompasses any biological unit capable of reproduction. The term “cell” contemplates both prokaryotic and eukaryotic cells. For example, a cell can be a bacterial cell, a plant cell, an animal cell including human cells, a fungus hypha or yeast cell. In one particular embodiment, a cell is a microorganism which is associated under healthy or diseased conditions with an oral biofilm. Non-limiting examples of cells include Bacteroides sp., Campylobacter rectus, Candida albicans, Capnocytophaga gingivalis, Centipeda periodontii, Citrobacter sp., Clostridium difficile, Corynebacterium matruchotii, Enterobacter cloacae, Enterococcus faecalis, Fusobacterium nucleatum, Hemophilus parainfluenzae, Klebsiella pneumoniae, Lachnospiraceae g.sp., Lactobacillus sp., Peptococcus prevoti, Peptostreptococcus anaerobius, Porphyromonas endodontalis, Porphyromonas gingivalis, Prevotella intermedia, Prevotella loescheii, Prevotella melaninogenica, Propionibacterium sp., Selemonad aremidis, Stomatococcus mud, Stomatococcus mucilaginosus, Streptoccoccus infantis, Streptococcus cristatus, Streptococcus gordonii, Streptococcus mitis, Streptococcus mutans, Streptococcus pneumoniae, Treponema denticola, and Veillonella sp.

Herein the term “biofilm” refers to any assemblage of cells adhering to the inner surface of the flow cell and to one another. In one embodiment, the biofilm comprises cells which are microorganisms. The biofilm may be comprised of both living and dead cells embedded in the matrix. Typically cells of a biofilm are embedded within or associated with a self-produced matrix of extracellular polymeric substances. For example, the extracellular matrix can include polysaccharides, eDNA, and proteins.

### Channels

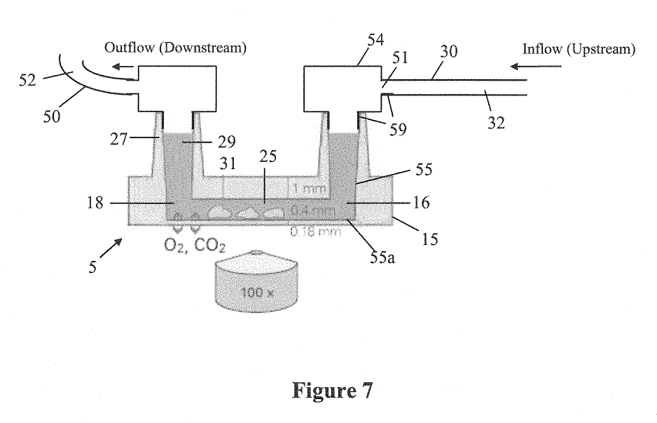

Referring to FIGS. 1 and 7, flow cell housing 5 can comprise a base 15 supporting a plurality of channel walls 55 defining a plurality of channels 25. Typically each channel 25 is a tube fluidly enclosed in cross-section with openings at either end. It will be understood that each individual channel 25 is typically defined by a single continuous channel wall 55. In some embodiments, the channel wall 55 can be a wall of a conduit which can be mounted to the surface of the base 15. For example, the conduit can be a hollow tube which can be removably or permanently mounted (e.g. with adhesive) to the surface of the base 15. Examples of materials making up the conduit are glass and plastic. In other embodiments, the channel walls 55 supported by the base 15 can be integral with the base 15. For example, the channel 25 can be defined by channel walls 55 formed by the material making up the base 15. Referring to FIG. 7, the material of the base 15 can define a bore having two openings on the same surface of the base 15. The bore openings can lead to chambers 29 defined by chamber walls 27 that descend into the interior of the base to connect with a channel 25 defined by channel walls 55.

In cross-section, the channel wall 55 can define any shape, examples of which include circular, oval, rectangular and hexagonal. The surface of a channel wall 55 defining a channel 25 can be smooth or rough. For example, the surface of the channel wall 55 can define ridges or small bumps that protrude into the channel 25. In the embodiment shown in FIG. 7, the channel walls 55 are straight and oriented substantially horizontal to define a substantially horizontal channel 25 without bends or curves. However, in other embodiments the channel wall 55 can bend, curve or zig-zag in a horizontal orientation. Further, in certain embodiments the channel wall 55 can have one or more upward or downward slopes to define a channel 25 that is sloped or undulating. The dimensions of a channel 25 defined by channel walls 55 can vary. In certain embodiments, a horizontally oriented channel 25 can have a height of less than 1 mm, a width of less than 5 mm, and a length of less than 25 mm. In certain embodiments, a horizontally oriented channel 25 can have a height of greater than 70 μm and a width of greater than 370 μm. In one non-limiting example, a horizontally oriented channel 25 can have a height of at least 400 μm, a width of at least 3.8 mm, and a length of 17 mm. In some embodiments, the channel 25 defined by the channel wall 55 can have a constant diameter or width along its length, whereas in other embodiments the channel 25 can vary in diameter or width along its length.

The surface of a channel wall 55 defining a channel 25 is typically configured to facilitate the formation and maintenance of a biofilm 31 (see FIG. 7). Accordingly, the surface of the channel wall 55 defining the channel 25 typically has properties which facilitate the adhesion of cells to the channels wall 55. In some embodiments, the surface of the channel wall 55 can be coated with one or more compounds which when applied to the channel wall 55 facilitate adhesion of cells. For example, the surface of the channel wall 55 defining the channel 25 can be coated with one or more compounds which when applied to the channel wall 55 result in the channel wall 55 exhibiting hydrophilic and/or adhesive properties that mediate the adhesion of cells. Compounds coating the surface of the channel wall 55 can be synthetic or natural (e.g. biological). Examples of materials coating the surface of the channel wall 55 include collagen I, collagen IV, fibronectin, poly-L-lysine, poly-D-lysine, mucin, hydroxyapatite, and filtered saliva.

Compounds can be applied to the surface of a channel wall 55 defining a channel 25 in any way known to a person of ordinary skill in the art. For example, solutions containing one or more compounds can be introduced into the channel 25 for a period of time to facilitate adhesion of the one or more compounds to the channel wall 55. The solution can be subsequently removed from the channel 25 (e.g. by aspiration) and the coated channel 25 then washed with an appropriate buffer. Alternatively, where the channels 25 are at least partly defined by the wall of a commercially available conduit, microscope slide or coverslip, the wall of the commercially available conduit, microscope slide or coverslip can be pre-coated with one or more compounds such that when the commercially available conduit, microscope slide or coverslip is incorporated into a flow cell 20, the channel wall 55 facilitates adhesion of microbes and thereby formation of a biofilm 31.

Referring to FIGS. 1 and 7, in some embodiments, a flow cell housing 5 can include one or more chamber walls 27 each defining a chamber 29 for receiving fluid. Typically each chamber wall 27 is adjacent to a channel wall 55 such that the chamber 29 is fluidly coupled to the channel 25. For example, as shown in FIG. 1, a channel 25 (e.g. the channel 25 labelled S1) can fluidly couple to a first chamber 29 (e.g. chamber “a”) positioned upstream of the channel 25, and fluidly couple to a second chamber 29 (e.g. chamber “b”) positioned downstream of the channel 25. Typically the chamber 29 has a greater cross-sectional area than a channel 25. Referring to FIG. 7, in some embodiments the chamber wall 27 can be integral with the base 15. For example, the interior surface of the chamber wall 27 defining the chamber 29 can be continuous with a channel wall 55 defining a channel 25. In such cases the material making up the chamber wall 27 is typically the same material as the base 15 (e.g. glass or plastic). Alternatively, for example in cases where the channel 25 is defined by a tube or conduit mounted to the base 15, the chamber wall 27 can be non-integral with respect to the base and can be made of the same material or different material than the material forming the base 15.

The number of channels 25 defined by a flow cell housing 5 of a continuous flow system 10 can vary. For example, in FIG. 1 the flow cell housing 5 defines 6 channels. In other embodiments the flow cell housing 5 can have 2-5 channels, or more than 6 channels.

### Base

Referring to FIG. 1, flow cell housing 5 can further include a base 15 to support channel walls 55 defining channels 25. The base 15 can be of any size, shape and material suitable to support channel walls 55 defining a plurality of channels 25. In one embodiment, the base 15 is a rectangle having the area dimensions of a standard microscope slide (i.e. about 75×25 mm), thickness 180 μm, and is composed of a material that meets optical requirements for microscopy (e.g. confocal microscopy). In certain embodiments, the base 15 is composed of glass or plastic.

As used herein, “around”, “about”, “approximately” or “substantially” shall generally mean within 20 percent, preferably within 10 percent, and more preferably within 5 percent of a given value or range. Numerical quantities given herein are approximate, meaning that the term “around”, “about”, “approximately” or “substantially” can be inferred if not expressly stated.

The base 15 can be configured to facilitate gas exchange between media contained within a channel 25 of the flow cell 20 and the ambient environment. For example, FIG. 7 illustrates a base 15 configured to facilitate the diffusion of oxygen and carbon dioxide across the channel wall 55 between the ambient environment and the channel 25. In certain embodiments, a base 15 facilitating gas exchange with the ambient environment can be obtained commercially. In one particular embodiment, a base 15 facilitating gas exchange is obtained by using the commercially available 6-channel ibidi™ μ-slide VI^{0.4} (ibidi GmbH Martinsried, Germany).

The plurality of channel walls 55 can be supported by the base 15 in any way known to an ordinary-skilled person. For example, the outer surfaces of channel walls 55 of conduits or tubes can be affixed to a top surface of the base 15 using an adhesive. In other embodiments (see FIG. 7) at least part of the plurality of channel walls 55 can be integral with the base 15 and/or embedded in the base 15.

In one embodiment, the base 15 defines a longitudinal axis (referenced by “L” in FIG. 1) extending between a first end of the base 15 (labelled “A” in FIG. 1) to a second end of the base 15 (labelled “B” in FIG. 1). For simplicity, FIG. 1 shows the longitudinal axis directed along the dimension of the base 15 which is longest, but it is recognized that the longitudinal axis can also run along the shortest dimension of the base (or, if the base is a square, can be arbitrarily defined).

The base 15 can further define a transverse axis (referenced by “T” in FIG. 1) that is oriented transverse to the longitudinal axis of the base 15 between a first side (labelled “C” in FIG. 1) and a second side (labelled “D” in FIG. 1) of the base 15. Herein the term “transverse” refers to an axis that is oriented perpendicular to the longitudinal axis or at any angle oblique to the longitudinal axis.

As is shown in FIG. 1, the plurality of channels 25 can be distributed adjacent to one another along the longitudinal axis of the base 15, with each of the plurality of channels 25 extending along a transverse axis of the base 15. In FIG. 1, the plurality of channels 25 extend parallel to one another along a transverse axis that is perpendicular with respect to the longitudinal axis of the base 15. In other embodiments, one or more of the plurality of channels 25 can extend at an angle that is oblique to the longitudinal axis.

One or more of the plurality of channels 25 can extend along one or more transverse axes between the first side (“C” in FIG. 1) and second side (“D” in FIG. 1) of the base 15. Herein the term “between the first side and second side” refers to the general direction of extension of the channels 25, and does not necessarily mean that the channels 25 extend all of the way to the edges of the base 15. In some embodiments (e.g. where each channel 25 is embedded in the base 15 and fluidly accessible via one or more chambers 29), one or more of the plurality of channels 25 extends between a first side and second side of the base 15 but terminate interior to the edges of the base 15.

Further, the relative angle of adjacent channels 25 along the longitudinal axis of base 15 can vary. For example, in FIG. 1 the channel walls 55 defining adjacent channels 25 are substantially parallel (i.e. all channels 25 are oriented perpendicular to the longitudinal axis). In other embodiments, channel walls 55 defining adjacent channels 25 can be arranged non-parallel along the longitudinal axis. For example, channel walls 55 of adjacent channels 25 can be supported by the base 15 such that individual channels are each oriented transversely with respect to the longitudinal axis but at different angles (e.g. a first channel can be oriented perpendicularly and a second channel can be oriented at an oblique angle with respect to the longitudinal axis of the base 15).

### Channel Connectors

Referring to FIGS. 1, 2 and 7, a continuous flow system 10 can include flow cells 20 comprising flow cell housing 5, multiple channel connectors 50 fluidly coupling adjacent channels 25 (e.g. via chamber walls 27 and adaptor 54), an upstream inflow conduit 30 connected to the flow cell housing 5 (e.g. via chamber walls 27 and adapter 54) to fluidly couple the plurality of channels 25 to an inflow channel 32 defined by the inflow conduit 30, and a downstream outflow conduit 40 connected to the flow cell housing 5 (e.g. via chamber walls 27 and adapter 54) to fluidly couple the plurality of channels 25 to an outflow channel 42 defined by the outflow conduit 40.

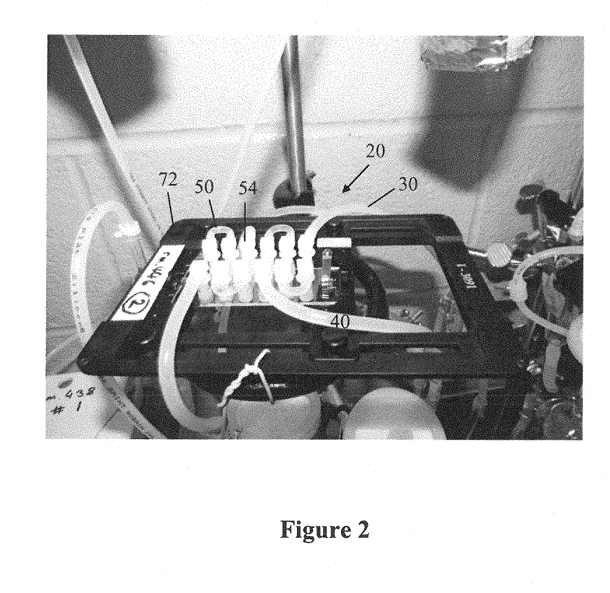

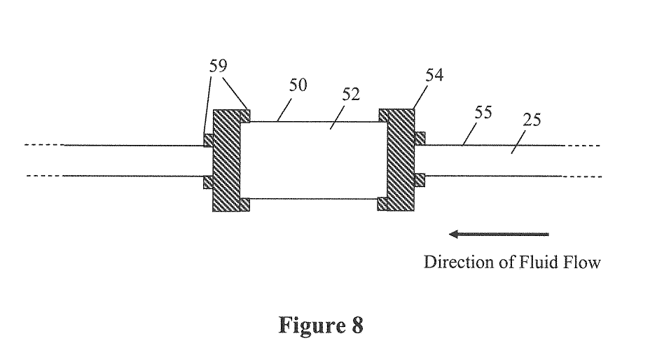

Each channel connector 50 can be a hollow tube fluidly enclosed in cross-section and open at either end. In certain embodiments, each channel connector 50 is shaped with one or more bends or curves. For example, as shown in FIG. 2, the channel connector 50 can be U-shaped. In other embodiments, each channel connector 50 can be straight. For example, in FIG. 8 a straight channel connector 50 fluidly couples adjacent channels 25 via connecting channel 52. The channel connector 50 can be reusable or disposable. For example, the channel connector 50 can be made of glass that is autoclavable such that the channel connector 50 can be sterilized between uses (i.e. in an embodiment where channel connectors 50 are removable from the flow cell housing 5). In other examples, the channel connector 50 can be made of plastic or another material that can be sterilized using chemicals or UV radiation. In certain embodiments the channel connector 50 is pre-sterilized and disposed of after use.

The connecting channel 52 defined by each channel connector 50 can be fluidly coupled at a first end to an outflow connection location 18 of an upstream channel 25 and fluidly coupled at a second end to an inflow connection location 16 of a downstream channel 25. The channel connector 50 can fluidly connect an upstream channel 25 and a downstream channel 25 in any way known to an ordinary-skilled person. For example, referring to FIGS. 1 and 7, a hollow first adaptor 54 (e.g. positioned in FIG. 1 at chamber “b”) can be sized to frictionally connect to a first chamber wall 27 defining a first chamber 29 fluidly coupled to the outflow connection location 18 of upstream channel 25 (e.g. channel S1 in FIG. 1). A portion of the side wall of the first adaptor 54 defining an opening 51 in the side wall can frictionally connect to a first end of the channel connector 50 to fluidly couple the connecting channel 52 to the outflow connection location 18 of the channel 25. A hollow second adaptor 54 (e.g. positioned in FIG. 1 at chamber “c”) can be sized to frictionally connect to a second chamber wall 27 defining a second chamber 29 fluidly coupled to the inflow connection location 16 of downstream channel 25 (e.g. channel S2 in FIG. 2). A portion of the side wall of the second adaptor 54 defining an opening 51 in the side wall can frictionally connect to a second end of the channel connector 50 to fluidly couple the connecting channel 52 to the inflow connection location 16 of the channel 25. In other embodiments, the connecting channel 52 can be fluidly coupled to one or more of an upstream outflow connection location 18 or a downstream inflow connection location 16 without the use of an adaptor 54. For example, each end of the connector 50 can be connected to a chamber wall 27 (or directly to a channel wall 55; see FIG. 8) using an adhesive.

The fluid coupling mediated by an adaptor 54 between channels 25 of the flow cell 20 can for example be by frictional (i.e. removable) engagement between the wall of the adaptor 54 and the chamber wall 27, the channel wall 55, or the wall of a channel connector 50. A wall of the adaptor 54 (e.g. via friction arm 59) can for example be configured to engage the inner surface of the chamber wall 27 (see FIG. 7) or the channel wall 55 (not shown). Alternatively, the inner surface of the wall of an adaptor 54 (e.g. via friction arm 59) can engage the outer surface of the chamber wall 27 (not shown) or the channel wall 55 (see FIG. 8). Likewise, the adaptor 54 can frictionally connect to a channel connector 50 via engagement of an inner surface of the wall of the adaptor 54 (e.g. via friction arm 59) to an outer surface of a wall of the channel connector 50 (see FIG. 8) or via engagement of an outer surface of the wall of the adaptor 54 (e.g. via friction arm 59 to an inner surface of a wall of the connector 50 (see FIG. 7). One advantage of using adaptors 54 which frictionally and removably connect to the channel connector 50 and chamber wall 27 (or channel walls 55) is that pre-sterilized disposable adaptors 54 can be used to facilitate an aseptic coupling of adjacent channels 25. In one particular embodiment, the removable adaptor 54 comprises a luer-lock plug. In other embodiments, the adaptor 54 can be fixedly mounted to one of the components of a flow cell 20 (e.g. fixedly mounted to the chamber wall 27 or fixedly mounted to a connector 50).

Channel connectors 50 can be used to fluidly couple any number of channels 25 of a flow cell housing 5. For example, FIG. 1 illustrates an embodiment where a flow cell housing 5 defines six channels 25 grouped into two flow cells 20 by fluidly coupling S1 and S2 channels 25 by a connecting channel 52 defined by a channel connector 50 and fluidly coupling S2 and S3 channels 25 by a connecting channel 52 defined by a channel connector 50. In other embodiments, multiple channel connectors 50 can fluidly couple more than three channels 25 of a flow cell housing 5 into a single flow cell 20. In a variation of the embodiment shown in FIG. 1, for example, five channel connectors 50 can fluidly couple all six channels defined by the flow cell housing 5. In other examples a flow cell 20 can include only two channels 25 fluidly coupled by a single connecting channel 52 defined by a channel connector 50. Therefore, the continuous flow system 10 described herein confers a dynamically configurable channel 25 length via selected use of removable channel connectors 50. Accordingly, the present disclosure provides for the dynamic configuration of a total length of a channel 25 (i.e. comprising individual channels 25) in order to regulate the residency time of cells within the channel 25.

It will be understood that fluid connection of channel connectors 50 to a flow cell housing 5 can transform the flow cell housing 5 from a group of isolated channels 25 into a flow cell 20 defining a series of fluidly coupled channels 25 for facilitating the flow of fluid from an upstream position to a downstream position. For example, in FIG. 1, the flow path of fluid through a flow cell 20 can occur as follows: from an upstream source reservoir 80 (not shown in FIG. 1) downstream to inflow channel 32 defined by inflow conduit 30; from inflow channel 32 downstream into the “a” chamber 29; from the “a” chamber 29 downstream to the “b” chamber 29 via the S1 channel 25; from the “b” chamber 29 downstream to the “c” chamber 29 via connecting channel 52; from the “c” chamber 29 downstream to the “d” chamber 29 via the S2 channel 25; from the “d” chamber 29 downstream to the “e” chamber 29 via connecting channel 52; from the “e” chamber 29 downstream to the “f” chamber via the S3 channel 25; and from the “f” chamber downstream to the outflow channel 42 defined by outflow conduit 40.

As will be understood, the connecting channel 52 is defined by the interior surface of a wall of the channel connector 50. In certain embodiments, the interior surface of the wall of the channel connector 50 comprises a surface material which is less suitable to formation of a biofilm by cells introduced into the flow cell 20. Accordingly, the interior surface of the wall of the channel connector 50 defining connecting channel 52 can be made of a material (e.g. glass) that is different from the material on the interior surface of a channel wall 55, and thereby facilitate less biofilm growth per unit area per unit time relative to the interior surface of channel wall 55. In certain embodiments, the interior surface of the wall of the channel connector 50 facilitates less biofilm growth per unit area per unit time relative to the interior surface of channel wall 55 because the interior surface of the wall of the channel connector 50 is made of a material that is not conducive to biofilm formation while the interior surface of the channel wall 55 is made with a material that is conducive to biofilm formation. In certain embodiments, the interior surface of the wall of the channel connector 50 facilitates less biofilm growth per unit area per unit time relative to the interior surface of channel wall 55 because the interior surface of the wall of the channel connector 50 is not coated and the interior surface of the channel wall 55 is coated with a coating that is conducive to biofilm formation (e.g. with a coating exhibiting hydrophilic and/or adhesive properties). In certain embodiments, the interior surface of the wall of the channel connector 50 facilitates less biofilm growth per unit area per unit time relative to the interior surface of channel wall 55 because the interior surface of the wall of the channel connector 50 is coated with a coating that is not conducive to biofilm formation. For example, the interior surface of the wall of a channel connector 50 can be coated with a hydrophobic compound.

By providing for connectors 50 which are generally not amenable to biofilm formation (i.e. having walls with an interior surface facilitating less biofilm growth per unit area per unit time relative to the interior surface of channel wall 55), the present disclosure provides the further advantage of connectors 50 which can be reused with ease to configure a channel 25 of a desired length. That is, by using connectors 50 having interior surfaces which discourage/do not facilitate adhesion and growth of cells introduced into a flow cell 20 incorporating the connectors 50, the connectors 50 can be easily removed from the flow cell 20, cleaned, sterilized (e.g. by autoclaving) and re-incorporated into fresh flow cells 20.

Typically the cross-sectional profile of a connecting channel 52 through a channel connector 50 is different than the cross-sectional profile of a channel 25 through a channel wall 55. In particular, typically a connecting channel 52 is of a greater cross-sectional area than a channel 25. For example, a channel connector 50 can have inner diameter 1.6 mm and be 32 mm long. By providing for a larger cross-sectional area of the connecting channel 52 relative to the channel 25, the present disclosure facilitates ease of handling of the removable connectors 50 when removing, cleaning, autoclaving, and reusing them.

Further, when a channel connector 50 fluidly couples adjacent channels 25, in certain embodiments the connecting channel 52 defined by the channel connector 50 is oriented in a different plane than the channels 25. For example, in certain embodiments the channel connector 50 when fluidly coupling adjacent channels 25 can define an arc that is not parallel with the plane of the channels 25 defined by channel walls 55. For example, the arc defined by the channel connector 50 when fluidly coupling adjacent channels 25 can be at an angle that is greater than 0° and equal to or less than 90° with respect to the plane of the channels 25.

Typically each channel 25 of a flow cell 20 is fluidly coupled by a connecting channel 52 directly to an adjacent channel 25. However, the present disclosure contemplates that channels 25 that are not directly adjacent along a longitudinal axis of the base 15 can be directly fluidly coupled by a connecting channel 52. For example, in FIG. 1, a channel connector 50 can directly fluidly couple the “b” chamber 29 to the “e” chamber 29, thereby enabling fluid in the flow cell 20 to bypass the S2 channel 25.

Further, contemplated herein is a continuous flow system 10 where a channel 25 can be directly fluidly coupled to more than one other channel 25. For example, the flow path of fluid through channels 25 of a flow cell 20 can be regulated by fluidly connecting a 3-way stopcock (not shown) to a chamber wall 27 such that one opening of the stopcock is fluidly coupled to a channel 25 (e.g. via a chamber 29) and the remaining two openings of the stopcock are each fluidly coupled to a different connecting channel 52 defined by two respective channel connectors 50. Each channel connector 50 can in turn be fluidly connected (e.g. by a frictional fit) to a different chamber wall 27, or alternatively to a second 3-way stopcock. For example, the chamber wall 27 defining the “b” chamber 29 in FIG. 1 can be fluidly connected to a 3-way stopcock such that a first nozzle of the 3-way stopcock can fluidly connect to a first channel connector 50 linking to the chamber wall 27 defining the “c” chamber 29. A second nozzle of the 3-way stopcock can fluidly connect to a second channel connector 50 linking to the chamber wall 27 defining the “e” chamber 29. The chamber wall 27 defining the “e” chamber 29 can in turn be fluidly connected to a second 3-way stopcock such that a first nozzle of the second 3-way stopcock fluidly connects to the second channel connector 50 originating at the chamber wall 27 defining the “b” chamber 29, while a second nozzle of the second 3-way stopcock can fluidly connect to a third channel connector 50 linking to the chamber wall 27 defining the “d” chamber 29.

As will be understood, by regulating the position of the valves of the first 3-way stopcock, the flow of fluid in the flow cell 20 can be directed alternately between the different connecting channels 52 defined by the different channel connectors 50 attached to each nozzle of the 3-way stopcock. For example, the valves of the 3-way stopcock fluidly connected to the chamber wall 27 defining the “b” chamber 29 can be positioned to facilitate the flow of fluid through the first channel connector 50 into the “c” chamber 29, or alternately the valves can be reversed to facilitate the flow of fluid via the second channel connector 50 into the “e” chamber 29. Where the valves are positioned to facilitate the flow of fluid to the “e” chamber 29, the valves of the second 3-way stopcock fluidly connected to the chamber wall 27 defining the “e” chamber 29 can also be positioned to facilitate the flow of fluid from the “b” chamber 29 into the “e” chamber 29. Further, given the nature of a 3-way stopcock to inhibit flow to the second nozzle when receiving flow from the first nozzle, the fluid entering the “e” chamber 29 via the second channel connector 50 is inhibited from back-flowing via the third channel connector 50 to the “d” chamber 29. Therefore, the flow path of fluid in the flow cell 20 can be regulated by the use of valved 3-way stopcocks fluidly connected to channel connectors 50.

Therefore, it will be understood that one or more channel connectors 50 for fluidly coupling channels 25 can be removable from the flow cell housing 5. Providing for removable fluid connections between multiple channels 25 of a flow cell housing 5 confers flexibility to a flow cell 20 in order to generate multiple alternate flow paths for fluid. In a further example, channel walls 55 defining two or more adjacent channels 25 can be seeded with an identical fluid containing cells (e.g. provided by a hydraulic pump 70; see FIG. 6) by fluidly coupling the channels 55 with one or more channel connectors 50 during seeding. Following seeding, at least one channel connector 50 can be removed so that at least two of the channels 25 are no longer fluidly coupled. Instead, each of the two seeded channels 25 can be coupled to one or more outflow reservoirs 81 (e.g. via outflow conduits 40). Fluids with different compositions (e.g. a control fluid and a fluid containing a molecular probe) can then be introduced into the uncoupled channels 25 from different source reservoirs 80 (e.g. different syringes) via different inflow conduits 30 to execute controlled experiments which assess the effects of one or more compounds on the biofilms.

### Inflow and Outflow

Referring to FIG. 1, a flow cell 20 of the continuous flow system 10 can further include an inflow conduit 30 defining an inflow channel 32 and outflow conduit 40 defining an outflow channel 42. An end of the inflow conduit 30 can be fluidly connected to a chamber wall 27 or channel wall 55 thereby facilitating the introduction of fluid into the chamber 29 and channel 25 via the inflow channel 32. In embodiments which do not include chambers 29 (see FIG. 8), the inflow conduit 30 can be directly fluidly connected to the channel wall 55. Likewise, an outflow conduit 40 can be fluidly connected to the flow cell 20 by fluidly connecting the outflow conduit 40 to a chamber wall 27 thereby facilitating the removal of fluid from the chamber 29 and channel 25 via outflow channel 42. In embodiments which do not include chambers 29, the outflow conduit 40 can be directly fluidly connected to the channel wall 55.

In certain embodiments, each of the inflow conduit 30 and the outflow conduit 40 can comprise tubing made of one or more materials which are flexible. For example, each of the inflow conduit 30 and outflow conduit 40 can be a flexible tube made of polyurethane, nylon, PVC, polyethylene, or silicone. In one particular embodiment, the inflow conduit 30 and outflow conduit 40 can each be a flexible tube comprising silicone. For example, the inflow conduit 30 and outflow conduit 40 can comprise Manosil® silicone tubing (Thermo-Fisher, Waltham, Mass., USA).

The inflow conduit 30 and outflow conduit 40 can be fluidly connected to the flow cell housing 5 (e.g. via a chamber wall 27 or channel wall 55) in any way known to an ordinary-skilled person. For example, referring to FIGS. 2 and 7, an adaptor 54 can be sized to frictionally connect to the chamber wall 27. An opening 51 on a side wall of the adaptor 54 can in turn fluidly connect to an end of the inflow conduit 30 or outflow conduit 40. As described above, with respect to the connection of the adaptor 54 to a chamber wall 27 or channel wall 55, in certain embodiments the outer surface of the wall of the adaptor 54 can be configured to frictionally connect to the inner surface of the chamber wall 27 (or alternatively the surface of the channel wall 55). In other embodiments the inner surface of the wall of the adaptor 54 can fluidly connect to the outer surface of the chamber wall 27. Likewise, an adaptor 54 can frictionally connect to the inflow conduit 30 and/or outflow conduit 40. One advantage of using adaptors 54 which frictionally and removably connect to the inflow conduit 30/outflow conduit 40 and chamber wall 27 is that pre-sterilized disposable adaptors 54 can be used to facilitate an aseptic fluid coupling of channels 25 defined by the flow cell 20 and the inflow channel 32 and outflow channel 42 defined by the inflow conduit 30 and outflow conduit 40, respectively. In one particular embodiment, the removable adaptor 54 comprises a luer-lock plug. In other embodiments, the adaptor 54 can be fixedly mounted to one of the components (e.g. fixedly mounted to the chamber wall 27 or fixedly mounted to the inflow conduit 30 or outflow conduit 40).

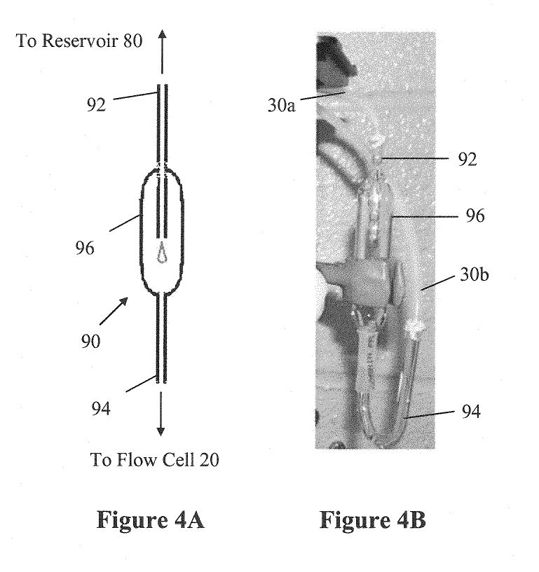

Referring to FIGS. 4A and 4B, in certain embodiments, the continuous flow system 10 can be equipped with a flow interrupter 90. The flow interrupter 90 can be positioned on the inflow side of the continuous flow system 10 (i.e. in FIG. 4B, between inflow conduit 30a and inflow conduit 30b) to inhibit contamination of fresh fluid contained in or flowing from a reservoir 80 with fluid backflowing from a channel 25 through a downstream inflow conduit 30b. In other words, the flow interrupter 90 can inhibit the contamination of fresh fluid (e.g. from a source reservoir 80) by fluid flowing upstream from a channel 25. In one embodiment, a flow interrupter 90 can comprise an inlet tube 92 fluidly connected to a body 96, which is fluidly connected to an outlet tube 94. In certain embodiments (e.g. FIG. 4A), the inlet tube 92 can extend into the cavity defined by the body 96. The inlet tube 92 and outlet tube 94 can be of various lengths and can be straight or have one or more bends (e.g. see FIG. 4B where the outlet tube 94 has a U-shaped bend). The inlet tube 92 can be fluidly connected to an end of an upstream inflow conduit 30a, which can be fluidly connected at its other end to the source reservoir 80 for distributing the fresh fluid. The outlet tube 94 of the flow interrupter 90 can in turn be fluidly connected to an end of a downstream inflow conduit 30b, which can fluidly connect at its other end to the flow cell housing 5 (e.g. at a chamber wall 27).

As shown in FIG. 4A, fluid (e.g. from the source reservoir 80) can flow into the flow interrupter 90 via one end of the inlet tube 92, from where the fluid can flow into the body 96 of the flow interrupter 90 through the opposing end of the inlet tube 92. The fluid can then enter the outlet tube 94 by gravity flow. As will be understood, in the event of the backflow of fluid (e.g. from a flow cell 20) into the outlet tube 94 of the flow interrupter 90, the backflowing fluid is inhibited from contacting the fresh fluid in the inlet tube 92 due to the space in the cavity of the body 96 between the opening of the outlet tube 94 into the body 96 and the tip of the inlet tube 92 extending into the body 96 for depositing the fresh fluid. In one embodiment, the space in the cavity of the body 96 between the tip of the inlet tube 92 depositing the fluid into the body 96 and the opening of the outlet tube 94 can be increased or decreased by adjusting the length of the inlet tube 92 which extends into the cavity of the body 96. For example, in FIG. 4A, the walls of the opening of the body 96 for receiving the inlet tube 92 can be lined with a gasket that frictionally connects to the inlet tube 92 and stabilizes the position of the inlet tube 92 during use of the flow interrupter 90. The length of the inlet tube 92 extending into the body 96 can then be adjusted by applying force on the inlet tube 92 either towards or away from the body.

In one embodiment, the flow interrupter 90 is reusable. For example, the flow interrupter 90 can be made of glass that is autoclavable such that the flow interrupter 90 can be sterilized between uses. In other examples, the flow interrupter 90 can be made of plastic or another material that can be sterilized using chemicals or UV radiation. In other embodiments the flow interrupter 90 is not reusable. For example, the flow interrupter 90 can be pre-sterilized and disposable.

Therefore, use of a flow interrupter 90 further lessens the likelihood that fluid from a source reservoir 80 will be contaminated by fluid backflowing from a channel 25. Other aspects of the continuous flow system 10 which reduce the likelihood of contamination of the system 10 include the use of components which are autoclavable, such as channel connectors 50, flow interrupter 90, and reservoirs 80, 81.

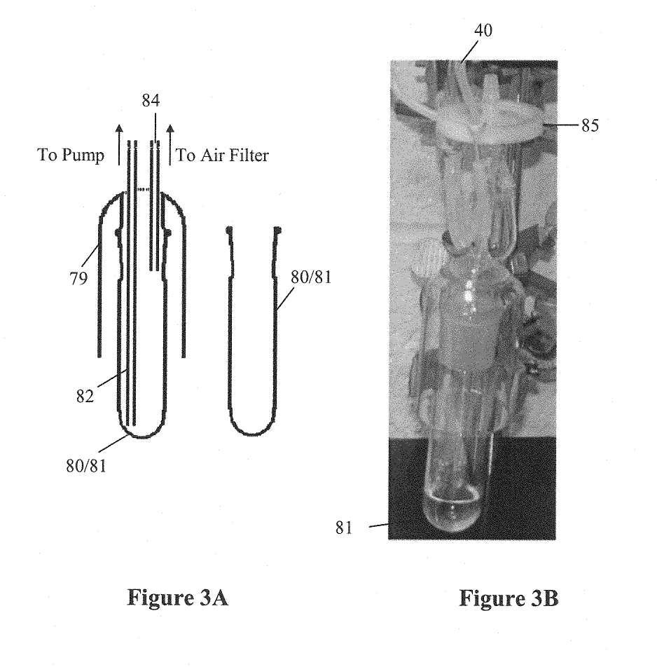

The likelihood of accidental air-borne contamination of the continuous flow system 10 (e.g. during supply of fresh nutrient media to the source reservoir 80, removal of used media from the outflow reservoir 81, or when using a source reservoir 80 that is open to the atmosphere) can further be reduced by applying a shield 79 to cover and shield a joint (i.e. opening) of a reservoir 80, 81 with the outer surface of the shielding. Such an arrangement is shown in FIG. 3A, where a shield 79 is configured as a glass bell shielding the reservoir 80. Typically the shield 79 is composed of glass which can be autoclaved to promote sterility.

### Reservoirs 80, 81 and Hydraulic Pump

Referring to FIGS. 3A, 3B and 6, the continuous flow system 10 can further include one or more reservoirs 80, 81 for containing fluid. For example, a source reservoir 80 can be positioned on the inlet side (i.e. upstream) of a flow cell 20 for supplying fresh fluid (e.g. microbial culture or sterile nutrient medium) to the flow cell 20 via an inflow conduit 30. An outflow reservoir 81 can be included on the outlet side (i.e. downstream) of the flow cell 20 for receiving fluid from the flow cell 20 via an outflow conduit 40. In one embodiment, one or more of the reservoirs 80, 81 are reusable and replaceable. For example, the reservoir 80, 81 can be made of glass that is autoclavable such that the reservoir 80, 81 can be detached from the connecting tubing and sterilized between uses. In other examples, the reservoir 80, 81 can be made of plastic or another material that can be sterilized using chemicals or UV radiation. In further embodiments the reservoir 80, 81 is not reusable. For example, the reservoir 80, 81 can be pre-sterilized and disposable. As described further below, in certain embodiments, the source reservoir 80 can be a syringe.

In certain embodiments, the source reservoir 80 can be open to the environment. As described, above, the likelihood of accidental air-borne contamination of the source reservoir 80 can be reduced by applying a shield 79 to cover and shield a joint (i.e. opening) of a source reservoir 80 with the outer surface of the shielding. Such an arrangement is shown in FIG. 3A, where a shield 79 is configured as a glass bell shielding the reservoir 80. Typically the shield 79 is composed of glass which can be autoclaved to maintain its sterility.

The source reservoir 80 can fluidly connect to an inflow conduit 30 to supply fluid to inflow channel 32 and thereby to flow cell 20. In one embodiment, the source reservoir 80 fluidly connects to the inflow conduit 30 via a hydraulic pump 70. For example, a reservoir tube 82 fluidly coupled to fluid in the source reservoir 80 (see FIG. 3A) can convey fluid from the reservoir 80 to the hydraulic pump 70, which can pump the fluid (e.g. via a flow interrupter 90) to the inflow conduit 30 fluidly connected to the flow cell 20. Typically, the source reservoir 80 is protected by an air filter 85 via air filter tube 84. In one particular embodiment, the air filter 85 can be a HEPA filter.

The outflow reservoir 81 can fluidly connect to outflow conduit 40 to receive fluid from the outflow channel 42 of flow cell 20. For example, FIG. 3B depicts an outflow reservoir 81 for receiving fluid from an outflow channel 42 defined by outflow conduit 40. Typically it is a good practice to protect the ambient atmosphere from possible aerosols coming from the outflow reservoir 81 using an air filter 85. In one particular embodiment, the filter 85 can be a HEPA filter.

Referring to FIG. 6, the continuous flow system 10 can further include a hydraulic pump 70 as a delivery system for precisely metering fluid received (e.g. via reservoir tube 82) from the source reservoir 80 to the inflow conduit 30 of a flow cell 20. In one particular embodiment, the hydraulic pump 70 is a peristaltic pump (e.g. Econo Gradient™ Pump #731-9001; Bio-Rad Laboratories, Ltd, Hercules, Calif.). Use of a hydraulic pump 70 (e.g. a peristaltic pump) in combination with the flow cells 20 described herein facilitates the emulation of hydraulic conditions suitable to oral bacteria. In particular, where the dimensions of a channel 25 approximate a height of 400 μm, a width of 3.8 mm, and a length of 17 mm, a small volume of fluid can be introduced to a flow cell 20 while maintaining a constant rate of flow by operation of the hydraulic pump 70 (e.g. peristaltic pump). In certain flow channels which have a smaller cross-sectional profile or a significantly smaller cross-sectional profile (e.g. 60× smaller), capillarity phenomena and/or hydrophobicity of channel walls can variably override the constant pressure of a pump (e.g. manostatic pump). One or more flow cells 20 can be metered fluid by a hydraulic pump 70. For example, while FIG. 6 shows a single flow cell connected to the hydraulic pump 70, in other embodiments the hydraulic pump 70 can service additional flow cells 20 (e.g. four flow cells 20 simultaneously).

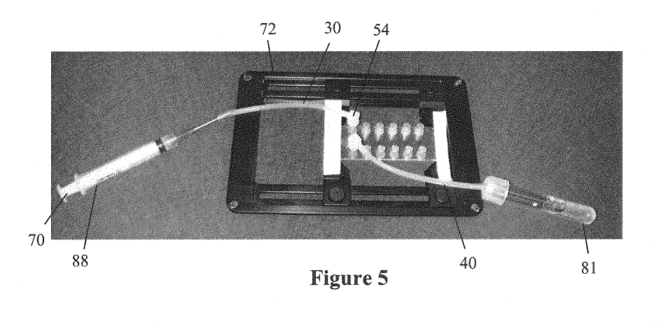

Referring to FIG. 5, in certain embodiments, the hydraulic pump 70 can be a manual pump. For example, hydraulic pump 70 can be a disposable syringe 88 fluidly connected to an end of inflow conduit 30 (e.g. comprising silicone tubing) via a blunt needle. Therefore, in such embodiments, the syringe 88 can act as both the hydraulic pump 70 and the reservoir 80. The other end of the inflow conduit 30 can be fluidly connected to a chamber wall 27 (e.g. via an adaptor 54) or directly to channel walls 55. Actuation of the hydraulic pump 70 (i.e. by pressing the plunger of the syringe 88) can result in fluid flowing from the syringe 88 through the blunt needle and inflow conduit 30 and into the channel 25 via chamber 29. As shown in FIG. 5, in certain embodiments (e.g. when seeding a channel wall 55 with cells), an opposing end of the channel 25 can be fluidly coupled via an outflow conduit 40 to an outflow reservoir 81 for receiving the fluid from the channel 25. In other embodiments, an opposing end of the channel 25 can be fluidly coupled to an adjacent channel 25 of the flow cell housing 5 via a channel connector 50.

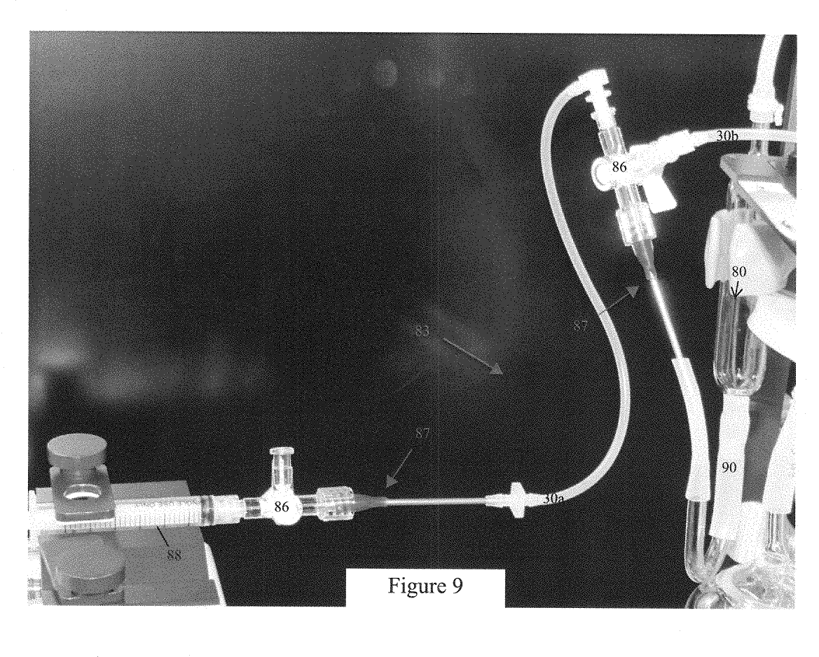

Referring to FIG. 9, in a further embodiment, the hydraulic pump 70 can be a linear pump used in combination with a syringe 88. For example, a linear pump can be used to deliver fluid to a channel 25 in a controlled manner. In one particular embodiment, the linear pump can be used in combination with two 3-way stopcocks 86, sterile disposable syringe 88 (acting as a source reservoir) and blunt needles 87 as a side-flow attachment 83 to deliver small amounts of fluid to channels 25 of a flow cell 20. As shown in FIG. 9, the side-flow attachment 83 (e.g. aseptically assembled in a biosafety cabinet) can be inserted downstream of the flow interrupter 90 and upstream of the flow cell 20.

In operation, the first 3-way stopcock can be open between the syringe 88 (acting as reservoir 80) and upstream inflow conduit 30a. The second 3-way stopcock 86 can be open between the upstream inflow conduit 30a and downstream inflow conduit 30b extending to the flow cell 20. By placing the syringe in the linear pump and exerting force on the plunger of the syringe 88 via the linear pump, a constant rate of fluid can be administered to the upstream inflow conduit 30a and thereby to the downstream inflow conduit 30b via the second stopcock 86 and to the channel 25 (not shown). For example, a flow rate of 0.2 ml/min of fluid can be administered for 10 minutes. After delivering the contents of the syringe, the first stopcock 86 can be closed and the flow of fluid (e.g. nutrient media) resumed by switching the second 3-way stopcock to connect the outflow from source reservoir 80 (i.e. via flow interrupter 90) to the flow cell 20. The vertical outlet of the first 3-way stopcock can be used for releasing pressure during the fill of the extension tubing with nutrient medium before the use of syringe 88. When used repeatedly during one assay, this outlet may be protected by an air filter 85 (e.g. HEPA filter). In one particular embodiment, the linear pump is a Fusion Touch Pump capable of outflow at a rate of between to 0.0001 μl/min to 102 ml/min and the 3-way stopcock withstands 200 psi of pressure. The linear pump, 3-way stopcocks and syringe are available for example from SAI Infusion Technologies, Illinois, USA. It will be understood that when two alternative pumps are operated on the opposite sides of a 3-way stopcock (e.g. linear vs. peristaltic) caution should be used not to actuate both pumps simultaneously.

A hydraulic pump 70 can be used to deliver any type of fluid to a flow cell 20. For example, the hydraulic pump 70 can deliver a fluid containing cells for seeding channel walls 55 with cells for forming a biofilm (i.e. inoculation of channels 25 by culture flow). Inoculation of a flow cell 20 by culture flow lessens the likelihood of contamination during seeding compared to a system which requires manual seeding of a surface (e.g. by pipetting fluid directly into a channel or into a reservoir connected to a channel). This is especially the case for systems which are implemented in a microplate and require removal of a lid of the microplate to access and seed an interior surface of the microplate, since it is well-known that removal of the lid of the plate and the movements of a user pipetting liquid into the plate makes the microplate vulnerable to contamination of the seeded cells. In contrast, by providing for a continuous flow system 10 which is fluidly enclosed during seeding, the risk of contaminating seeded cells is dramatically reduced.

In other embodiments, a linear pump in combination with a syringe can be used to deliver fluids containing molecules for probing a biofilm established on the interior surface of a channel wall 55. For example, the fluid can contain molecular probes (e.g. labelled with fluorescent or radioactive moieties) capable of recognizing and binding to molecular targets on the surface of or within cells of the biofilm, or in the extracellular matrix surrounding the cells of the biofilm. In other embodiments, the fluid can contain non-labelled molecules for contact with the biofilm in order to observe the response of the cells in the biofilm to the ingredients. Non-limiting examples of compounds contained in the fluid introduced into a channel by hydraulic pump 70 include one or more of proteins, amino acids, nucleic acids, sugars, polysaccharides, nucleosides, lipids, and drugs/pharmacological compounds.

The hydraulic pump 70 can be connected to the source reservoir 80 in any way known to a person of ordinary skill in the art. For example, the source reservoir 80 can be operably connected to the hydraulic pump 70 by reservoir tube 82, which can be made of one or more materials which are flexible, amenable to autoclaving, and have no known effect on microbial growth. For example, reservoir tube 82 can be a flexible tube made of silicone or Tygon. In one particular embodiment, the reservoir tube 82 can each be a flexible tube comprising silicone. For example, the reservoir tube 82 can comprise Manosil® silicone tubing (Thermo-Fisher, Waltham, Mass., USA).

Further, the connection between the reservoir tube 82 and hydraulic pump 70, and between hydraulic pump 70 and inflow conduit 30, can be facilitated in any way known to a person of ordinary skill in the art. In one particular embodiment, couplers purchased from Bio-Rad (Bio-Rad Laboratories, Ltd, Hercules, Calif.) are used.

It will be understood that the presently described continuous flow system 10 incorporates features which inhibit contamination of the system 10 while simultaneously providing for the ability to establish and monitor a biofilm containing defined species of cells (e.g. bacterial cells). For example, the continuous flow system 10 described herein facilitates a removable seeding mechanism which accommodates precise control over the quantity, type and timing of cells introduced into one or more channels 25 while inhibiting contamination of resulting biofilms. For example, by providing for a removable side-flow attachment 83 (see FIG. 9), defined quantities and types of cells can be aseptically introduced into one or more channels 25 without the need to pipette the cells directly into the system, thereby inhibiting contamination of resulting biofilms. For example, a known quantity of a first species of cells can be inoculated into one or more channels 25 using a first side-flow attachment 83, followed by inoculation of a known quantity of a second species of cells using a second side-flow attachment 83, to examine the effect of the timing of introduction of particular cell types in a mixed inoculum on resulting biofilm formation and composition.

Contamination of the continuous flow system 10 is further inhibited by providing a source reservoir 80 which can supply fresh and sterile fluid (e.g. nutrient medium) to channels 25 without the need to directly access the reservoir 80 or channels 25 (e.g. by pipetting fresh medium directly into the system) to regulate the flow of the fluid. For example, as described above, a hydraulic pump 70 can be used to precisely regulate the flow rate of fluid into the channels 25. In addition, when employing a side-flow attachment 83, a stopcock can be used to temporarily halt the flow of fluid from a source reservoir 80 in order to facilitate flow of fluid into the channels 25 from a syringe 88. Configuring the source reservoir 80 to be removable from the continuous flow system 10 further facilitates aseptic control over the content of fluid introduced into channels 25. For example, a first source reservoir 80 can provide a first nutrient medium to channels 25 for a set period of time, following which a second source reservoir 80 can be used as a source of a second nutrient medium into channels 25. In certain embodiments, the second source reservoir 80 can be a sterile syringe 88 containing dyes or fluorescent probes for examining biofilm activity.

### Operation

It will be understood that the continuous flow system 10 described herein operates by passing fluid through the flow system 10 from upstream positions to downstream positions via a particular flow path. In one embodiment a flow path is defined by the flow of fluid from the upstream source reservoir 80 to the downstream outflow reservoir 81. For example, the flow path can define the flow of fluid from the source reservoir 80 downstream to the hydraulic pump 70 (e.g. via the tube 82 fluidly coupled to fluid in the source reservoir 80) downstream to the flow interrupter 90, downstream to the inflow channel 32 defined by the inflow conduit 30, downstream to the chamber 29 defined by the chamber wall 27, downstream to a channel 25 defined by channel wall 55, downstream to a connecting channel 52 defined by a channel connector 50, downstream to one or more further channels 25 defined by channel walls 55, each of the one or more further channels 25 fluidly connected by a further connecting channel 52 defined by a further channel connector 50, downstream to an outflow channel 42 defined by an outflow conduit 40, downstream to the outflow reservoir 81. In some embodiments, the flow interrupter 90 can be absent. In other embodiments, the source reservoir 80 and the hydraulic pump 70 can be consolidated, such as when a syringe is used to both contain the fluid and pump the fluid into a channel 25 via one or more inflow conduits 30.

Typically, prior to use of a continuous flow system 10, the temperature of the flow cell 20 and/or fluid (e.g. seeding medium and/or nutrient media) is equilibrated to a temperature (e.g. 37° C.) compatible with the seeding and growth of a biofilm. Referring to FIGS. 5 and 6, the continuous flow system 10 can be operated by first aseptically introducing cells into one or more channels 25 to facilitate formation of one or more biofilms on the interior surface of channel walls 25. For example, as shown in FIG. 5, channel walls can be seeded with cells by using a hydraulic pump 70 (e.g. syringe) to pump a fluid containing the cells into a chamber 29 via inflow conduit 30. As will be understood, cells introduced into a channel 25 can adhere to a surface of the channel wall 55 to form a biofilm. As described above, formation of a biofilm on the surface of the interior channel wall 55 can be facilitated by including a coating (e.g. exhibiting hydrophilic and/or adhesive properties) on the interior surface of the channel wall 55. In certain embodiments, the fluid can be collected from the inoculated channel 25 by fluidly connecting an outflow reservoir 81 to a chamber wall 27 positioned a portion of the channel 25 downstream to the connection of the inflow conduit 30. In other embodiments, the inoculated fluid can flow to one or more downstream channels 25 via channel connectors 50 fluidly connected to chamber walls 27 (or alternatively fluidly connected directly to channel walls 55). As shown in FIG. 5, in some embodiments the fluid containing cells for seeding one or more channels 25 of a flow cell 20 can be contained in a syringe which also functions as a hydraulic pump 70. In other embodiments, the fluid containing cells for seeding the flow cell 20 can be provided by a non-syringe source reservoir 80 (see FIG. 6).

Once channel walls 55 have been seeded with cells to establish a biofilm, the biofilm can be treated or manipulated in various ways. For example, in embodiments (e.g. FIG. 5) in which only a single channel 25 is seeded, outflow conduit 40 and outflow reservoir 81 can be disconnected from the chamber wall 27 at the downstream end of the seeded channel 25 and one a channel connector 50 can be used to fluidly couple the seeded channel to a second downstream channel 25. In certain embodiments (e.g. where the hydraulic pump facilitating seeding of channel 25 is a syringe), the hydraulic pump 70 can be replaced following seeding. For example, inflow conduit 30 can be disconnected from the blunt needle of the syringe and fluidly connected to a source reservoir 80 (e.g. via flow interrupter 90) in turn connected to an automatic hydraulic pump. Alternatively, switching between seeding and flow of nutrient medium can occur via manipulation of flow through three-way stopcocks, as described above. In either event, the source reservoir 80 can contain fluid (e.g. sterile nutrient medium) which can be delivered to the flow cell 20 via one or more inflow conduits 30.

By providing for removable connectors 50 to fluidly couple channels 25 adapted to support biofilm growth, the present continuous flow system 10 facilitates flexible and adaptable seeding of channel walls 55. For example, as shown in FIG. 5, a user can seed a channel wall 55 of only a single channel 25 by fluidly connecting an outflow reservoir 81 to a chamber wall 27 downstream of the chamber 29 receiving the seed from the hydraulic pump 70. If subsequent to seeding the seeded channel 25 is fluidly coupled to one or more adjacent channels by channel connectors 50, then a user can subsequently administer nutrient medium to the flow cell 20 to monitor movement of cells from the biofilm formed along the seeded channel walls 55 to adjacent channels 25 fluidly coupled to the seeded channel 25 by the connectors 50. Alternatively, a channel 25 receiving fluid containing cells from a hydraulic pump 70 (e.g. syringe) can be fluidly coupled to an adjacent channel 25 by a connector 50 during seeding. By fluidly coupling channels 25 during seeding, multiple biofilms can be formed simultaneously from an identical cell culture along channel walls 55 defining adjacent channels 25. The biofilms along channel walls 55 defining adjacent channels 25 can then subsequently be treated in controlled experiments with different compounds (i.e. contained in different fluids introduced independently to the adjacent channels 25 via one or more hydraulic pumps 70) to examine the response of cells in the biofilms to one or more compounds. For example, following seeding of adjacent channels 25 fluidly coupled during seeding by a channel connector 50, the channel connector 50 can be removed from chamber walls 27 and each chamber 29 can be fluidly coupled downstream of the seeded channel 25 to a different outflow conduit 40 leading to an outflow reservoir 81. Each of the seeded channels 25 can also be fluidly connected upstream of the channel 25 to a different inflow conduit 30 which can each receive fluid from a different source reservoir 80 (e.g. a syringe 88). In one embodiment, each source reservoir 80 contains fluid which differs from the fluid in the other source reservoir 80 by one or more compounds (e.g. fluorescent or radiolabelled probes). Since the seeded channels 25 are no longer fluidly coupled, controlled experiments can be executed to assess the effect of a particular compound on a biofilm by delivering into the different seeded channels 25 the different fluids containing the different one or more compounds. In one embodiment, the different fluids are delivered into the different channels 25 by inserting syringes 88 (i.e. source reservoir 80) containing the different fluids into one or more linear pumps (i.e. in combination with the syringe, hydraulic pump 70) and actuating the linear pumps.

As will be understood, following seeding of one or more channel walls 55 with cells to form a biofilm, the biofilm can be permitted to grow for an indefinite amount of time by providing the cells with nutrient medium delivered from source reservoir 80. A further advantage of the presently described continuous flow system is that the cross-sectional profile of channels 25 is amenable to the development of a biofilm comparable to that which exists in an oral cavity. In particular, the cross-sectional profile of each channel 25 is large enough (e.g. in one embodiment, a height of at least 400 μm and a width of at least 3.8 mm) to accommodate significant biofilm formation while maintaining shear stress and/or cell residency times at levels comparable to those in an oral cavity.

Referring to FIGS. 2 and 7, in certain embodiments, one or more flow cells 20 can be configured to be transportable to a stage of a microscope for imaging biofilms on the interior surface of channel walls 55. For example, prior to connecting a flow cell housing 5 to an inflow connector 30 and outflow connector 40, the base 15 of flow cell housing 5 can be inserted into a microscope stage insert 72. In a non-limiting example, the microscope stage insert is a Universal Insert 160×110 mm with retaining clips (Applied Scientific Instrumentation, Inc., Eugene, Oreg.). Cells of a biofilm (e.g. biofilm 31 in FIG. 7) formed along a channel wall 55 can then be visualized using a microscope (e.g. at 100× magnification) in a way known to a person of ordinary skill in the art. Typically visualization of biofilms is performed at the bottom of a channel wall 55 (i.e. channel wall 55a in FIG. 7), as it can be difficult to visualize biofilm organisms on the vertical walls, as high power lenses with short focal distance are needed. In some embodiments, the bottom of a channel wall 55 (i.e. channel wall 55a) consists of a thin microscope coverslip bonded to the bottom of base 15.

Based on previously published data collected in vivo, (cited below) the ranges of fluidic parameters of normal oral environment (during awake period) are known. Specifically, three aspects of fluidic conditions have been suggested to influence the biofilm growth, namely shear stress, fluid velocity and dilution rate. These parameters are correlated such that, for a particular cross-section of channel 25, increased fluid velocities tend to result in increased shear stress and increased dilution rate. Further, the parameters can be positively inter-correlated so that values higher than the normal range occur temporarily during meal consumption, values lower than the range occur only in certain regions of the mouth or during the night sleep. The present continuous flow system 10 facilitates regulation of shear stress, fluid velocity and dilution rate within channels 25 to advantageously simulate actual values of these parameters in oral cavities.

Moreover, as the cross-sectional area of the tubing in an oral simulator is reduced, the effect of excessive dilution rates and shear stress can be exacerbated by other physical phenomena, such as viscous forces dominating over inertial forces, and temperature and pressure-driven generation of micro-bubbles. Contrary to known WPM flow cells, a further advantage of the presently described system 10 is that it is well-suited for extending the range of fluidic parameters to lower or higher values due to a wide range of flow rates (e.g. 120 μL h⁻¹ to 2.4 L h⁻¹) available with the peristaltic pump (i.e. hydraulic pump 70) used. As a result, the system 10 can be used to simulate various manipulations to an oral biofilm, such as for example contact of the biofilm with an instrument (e.g. toothbrush), rinsing of the biofilm, and/or selecting microorganisms in the biofilm which have a relatively high capacity to adhere to the biofilm.

Shear stress (typical oral values of 0.0010-0.5 dyn·cm⁻²) drives adhesion and release of cells to and from a wetted surface (e.g. surface of a tooth or channel wall 55) capable of supporting biofilm formation. The present continuous flow system 10 can advantageously approximate in vivo values for shear stress. In contrast, published data from WPM flow cells are approximately 20× higher than the lower-bound values achieved with the system 10. In certain embodiments, shear stress values within a channel 25 of the presently described continuous flow system 10 can be 0.0024-3.00 dyn·cm⁻², preferably 0.0024-2.00 dyn·cm⁻², and more preferably 0.0024-0.36 dyn·cm⁻².

In certain embodiments, ranges of shear stress in a channel 25 of the system 10 differ depending on a stage of operation of the system 10. For example, at a first stage of operation the channel 25 can receive fluid (e.g. by the action of hydraulic pump 70 pumping the fluid into channel 25 via inflow conduit 30) exhibiting a shear stress value within a pre-determined first range of shear stress values. At a second stage of operation, the rate that the fluid is received into the channel 25 can be modified (e.g. by manually regulating the rate of pumping by hydraulic pump 70) such that the channel 25 receives fluid exhibiting a shear stress value within a pre-determined second range of shear stress values, the second range of shear stress values being outside of the first range of shear stress values. A first stage of operation of the system 10 can involve introducing (e.g. by the action of hydraulic pump 70 via inflow conduit 30) cells into a channel 25 of the system 10 at pre-determined shear stress values within a first range that facilitates attachment of the cells to a channel wall 55 (i.e. seeding the channel wall 55 to form a biofilm). This first stage of operation of the system 10 can simulate shear stress values in an oral cavity during “normal” periods of oral biofilm establishment and growth. A second stage of operation of the system 10 can involve introducing (e.g. by the action of hydraulic pump 70 via inflow conduit 30) nutrient media into the channel 25 at pre-determined shear stress values within a second range, which can be higher than and fall outside of the first range, in order to simulate forces which act in oral cavities to potentially disrupt an established biofilm (i.e. cause drifting of cells in the biofilm). For example, at the second stage, the second range of shear stress values can simulate shear stresses applied to an oral biofilm by an instrument such as a toothbrush when the instrument contacts the biofilm.

Non-limiting values defining the pre-determined first range of shear stress in the channel 25 during the first stage of operation are 0.0024-0.36 dyn·cm⁻², 0.024-0.5 dyn·cm⁻², 0.024-0.6 dyn·cm⁻², and 0.024-0.7 dyn·cm⁻². Non-limiting values defining the pre-determined second range of shear stress in the channel 25 during the second stage of operation are 0.36-0.5 dyn·cm⁻², 0.5-1.0 dyn·cm⁻², 0.5-2.0 dyn·cm⁻², 0.6-1.5 dyn·cm⁻² and 0.7-2.0 dyn·cm⁻².

Fluid velocity (typical oral values of 0.8-7.6 mm·min⁻¹) in the proximity of an in vivo oral biofilm is closely correlated with shear stress. The present continuous flow system 10 can advantageously approximate the upper bound of the in vivo range for fluid velocity. In contrast, WPM flow cells produce values for fluid velocity which exceed the lower-bound values achieved by the system 10 described herein by at least 20-fold. In certain embodiments, fluid velocity values within a channel 25 of the presently described continuous flow system 10 can be 1.32-1650 mm·min⁻¹, preferably 1.32-1100 mm·min⁻¹, more preferably 1.32-198 mm·min⁻¹, and most preferably 1.32-7.6 mm·min⁻¹.

In certain embodiments, ranges of fluid velocities in a channel 25 of the system 10 differ depending on a stage of operation of the system 10. For example, at a first stage of operation the channel 25 can receive fluid (e.g. by the action of hydraulic pump 70 pumping the fluid into channel 25 via inflow conduit 30) exhibiting a fluid velocity value within a pre-determined first range of fluid velocity values. At a second stage of operation, the rate that the fluid is received into the channel 25 can be modified (e.g. by manually regulating the rate of pumping by hydraulic pump 70) such that the channel 25 receives fluid exhibiting a fluid velocity value within a pre-determined second range of fluid velocity values, the second range of fluid velocity values being outside of the first range of fluid velocity values. A first stage of operation of the system 10 can involve introducing (e.g. by hydraulic pump 70 via inflow conduit 30) cells into a channel 25 of the system 10 at pre-determined fluid velocity values within a first range that facilitates attachment of the cells to a channel wall 55 (i.e. seeding the channel wall 55 to form a biofilm). This first stage of operation of the system 10 can simulate fluid velocity values resulting from for example normal or stimulated salivation in an oral cavity during periods of oral biofilm establishment and growth. A second stage of operation of the system 10 can involve introducing (e.g. by hydraulic pump 70 via inflow conduit 30) nutrient media into the channel 25 at pre-determined fluid velocity values within a second range, which can be higher than and fall outside the first range, in order to simulate forces which act in oral cavities on an established biofilm. For example, at the second stage, the second range of fluid velocity values can simulate fluid velocities present while rinsing a biofilm in an oral cavity (for example, with mouthwash).

Non-limiting values defining the pre-determined first range of fluid velocity in the channel 25 during the first stage of operation are 1.32-100 mm·min⁻¹, 1.32-150 mm·min⁻¹ and 1.32-200 mm·min⁻¹. Non-limiting values defining the pre-determined second range of fluid velocity in the channel 25 during the second stage of operation are 100-200 mm·min⁻¹, 150-500 mm·min⁻¹, 200-500 mm·min⁻¹, 200-1000 mm·min⁻¹ and 100-1500 mm·min⁻¹.

Dilution rate (typical oral values of 11.1-19.0 h⁻¹) exerts selection pressure on cells in suspension (plankton) as well as provides exchange of fluids in the proximity of a biofilm in an oral cavity. The present continuous flow system 10 can advantageously approximate the upper bound of the in vivo range of dilution rate. In contrast, WPM flow cells typically produce dilution rate values that exceed the lower-bound values achieved by the system 10 described herein by at least 6×. Dilution rates within a channel 25 of the presently described system 10 can be 4-5000 h⁻¹, preferably 10-3500 h⁻¹, more preferably 10-600 h⁻¹, and most preferably 11.1-19.0 h⁻¹.

In certain embodiments, ranges of dilution rates in a channel 25 of the system 10 differ depending on a stage of operation of the system 10. For example, at a first stage of operation the channel 25 can receive fluid (e.g. by the action of hydraulic pump 70 pumping the fluid into channel 25 via inflow conduit 30) exhibiting a dilution rate value within a pre-determined first range of dilution rate values. At a second stage of operation, the rate that the fluid is received into the channel 25 can be modified (e.g. by manually regulating the rate of pumping by hydraulic pump 70) such that the channel 25 receives fluid exhibiting a dilution rate value within a pre-determined second range of dilution rate values, the second range of dilution rate values being outside of the first range of dilution rate values. A first stage of operation of the system 10 can involve introducing (e.g. by hydraulic pump 70 via inflow conduit 30) cells into a channel 25 of the system 10 at pre-determined dilution rate values within a first range that facilitates attachment of the cells to a channel wall 55 (i.e. seeding the channel wall 55 to form a biofilm). This first stage of operation of the system 10 can simulate dilution rate values resulting from for example normal or stimulated salivation in an oral cavity during periods of oral biofilm establishment and growth. A second stage of operation of the system 10 can involve introducing (e.g. by hydraulic pump 70 via inflow conduit 30) nutrient media into the channel 25 at pre-determined dilution rate values within a second range, which can be higher than and fall outside of the first range, in order to simulate forces which act in oral cavities on an established biofilm. For example, at the second stage, the second range of dilution rate values can be pre-determined to simulate a fluid velocity value produced while rinsing a biofilm in an oral cavity (for example, with mouthwash). In other examples, the second range of dilution rate values can be pre-determined to select for species of microorganisms which are capable of adhering to a surface (i.e. channel wall 55) within the range of pre-determined dilution rate values (i.e. select against species of microorganisms which are incapable of adhering to the surface within the range of pre-determined dilution rate values). Therefore, by providing for a second range of dilution rate values outside of the first range of dilution rate values, the system 10 can select for particular species of microorganisms that adhere relatively strongly to a biofilm.

Non-limiting values defining the pre-determined first range of dilution rates in the channel 25 during the first stage of operation are 4-600 h⁻¹, 10-600 h⁻¹ and 11.1-600 h⁻¹. Non-limiting values defining the pre-determined second range of dilution rates in the channel 25 during the second stage of operation are 600-1000 h⁻¹, 600-3500 h⁻¹, and 600-5000 h⁻¹.

### Prototype Fluidic Features

We used published data on the normal un-stimulated saliva secretion (Elishoov et al. 2008, Arch. Oral Biol. 53:75) and the volume of saliva in mouth before swallowing (Lagelof & Dawes, 1984, J. Dent. Res. 63:618) and estimated the dilution rate prevailing in the human oral cavity during “awake period” as approximately D≦20 h⁻¹ (not calculated by the above authors). Compared with available data on maximum growth rates of bacterial species found in the human biome, this value is about 10× higher than bacteria can attain (as “planktonic cells”; data compiled from more than 12 published papers; see Table 1). Since both the oral cavity dilution rates and bacterial growth rates are expressed in the same physical units (h⁻¹), these data mean that the saliva flow greatly exceeds the potential of bacterial cells suspended in saliva to sustain their numbers (as “planktonic cells”).

TABLE 1
Maximum growth rates of bacterial species.

| | | Growth Rate | Doubling Time |
|---|---|---|---|
| Species | Temperature | h⁻¹ | min |
| Fastest species outside | | | |
| human biome: | | | |
| Pseudomonas natrigenes | 37° C. | 4.24 | 9.8 |
| Vibrio parahaemolyticus | 37° C. | 3.78 | 11 |
| Fastest species found in | | | |
| human biome: | | | |
| Escherichia coli | 37° C. | 2.08 | 20 |
| Klebsiella pneumoniae | 35° C. | 1.87 | 22 |
| Bacillus subtilis | 37° C. | 1.39 | 30 |

Our research of oral bacterial strains in continuous-flow systems during recent years (Legner & Cvitkovitch; unpublished data) showed that the rate of initial biofilm formation increases with the dilution rate of the fluids. In the light of the above published data, this suggests that the selection pressure proportional to the dilution rate of saliva gives a great advantage to the sedentary (surface attached) as compared to the suspended (planktonic) way of life of oral microorganisms.

With this information in mind, we assembled a continuous-flow system that would advantageously emulate oral cavity conditions for our strains and most importantly for any sample of oral microbiome freshly isolated from a patient's oral cavity (i.e., ex-vivo microbiome). A combination of the Ibidi Slide VI^{0.4} (i.e. base 15 containing channels 25 defined by channel walls 55) with Bio-Rad™ Econo Gradient pump (i.e. pump 70) allowed for a good approximation of the above-specified conditions. A single-channel 25 volume of Ibidi Slide (i.e. base 15) is 30 μL and the minimum flow rate of the pump 70 (with the inner diameter of pump tubing being 0.8 mm) is 600 μL per hour which results in D=20 h⁻¹, if considering a single channel 25 to be a continuous-flow vessel. While, arguably, prevailing laminar streaming in the channel 25 may cause substantially higher values of D at any given point of the channel 25, practical implementation of the system sustained massive biofilm formation of both single oral isolates and entire plaque samples (Wenderska, Legner, Cvitkovitch; Huang, Legner, Finer; unpublished data). Moreover, connecting the Ibidi channels 25 (using silicone tubing as channel connectors 50) in series of up to 6 units allowed us to visualize and quantify a gradient of biofilm and extracellular matrix mass decreasing from a single-point nutrient supply (inflow to the Channel 1) towards the outflow from the system (Channel 6).

Another parameter to watch for while simulating oral conditions is the shear stress under which oral biofilms (the plaque) exist. A shear stress during normal salivation is reported to be 0.001 to 0.5 dyn·cm⁻², while the shear stress during e.g. biting an apple (stimulated salivation) is known to be at 2.0 dyn·cm⁻² (Busher & van der Mei 2006; Clinical Microbiol. Rev. 19: 127).

When application to oral plaque conditions is attempted using the WPM flow cells (e.g. BioFlux™) systems, the effort is made to simulate actual shear stress values. However, a disadvantage of known systems is that the shear stress they generate only marginally overlaps with the normal salivation values (the lower bound of reported range for BioFlux™ is at 0.2 dyn·cm²; Nance et al. 2013; J. Antimicrob. Chemotherapy 68:2550). Contrary to known systems, the presently described continuous flow system 10 can advantageously give rise to a lower bound of available range of shear stress values of 0.01 dyn·cm⁻², which is well within normal oral salivation conditions.

Table 2 shows the results of a comparison of shear stress in the proximity of a submerged surface between Ibidi channels 25, a BioFlux™ system, and a rotating disc reactor.

The excessive downscaling of channel cross sections of known systems (e.g. BioFlux™) is associated with a substantial decrease of the Reynolds number (cf. Gupta 2014, Viscometry for Liquids: Calibration of Viscometers; Springer) so that the viscous forces begin to dominate over inertial forces (Purcell 1977; Amer. J. Physics 45:3). This is also exacerbated by other physical phenomena, such as Henry's Law (temperature and partial pressure driven generation of micro bubbles). As a result, in known systems (e.g. BioFlux™) the WPM flow cell applications for ex vivo plaque samples require constant microscope control to detect frequent obstructions to the flow. Contrary to these known systems, in the presently described continuous flow system 10, flow obstructions are rare providing for a more reliable simulation of oral biofilm conditions.

TABLE 2
Shear Stress τ in the proximity of submerged surface
Ibidi μ-Slide VI^{0.4} (per channel)

| | F (mL · | τ (dyn · | |
|---|---|---|---|
| D (h⁻¹) | h⁻¹) | cm⁻²) | |
| 20.0 | 0.6 | 0.012 | biofilm growing; no drifting effect |
| 3,333 | 100 | 2.0 | drift of S. mutans from Ch1 to Ch 6 |

BioFlux System (Nance et al., 2013)

| F (mL · | τ (dyn · | |
|---|---|---|
| h⁻¹) | cm⁻²) | |
| 0.018 | 0.2 | attached multi-species biofilm growing |
| 0.090 | 1.0 | pellicle coating; biofilm seeding |
| 0.74 | 8.0 | biofilm harvesting (at back-forth motion) |

Rotating Disc Reactor (Vinogradov et al., 2004)

| τ (dyn cm⁻²) | Viscoelastic Response |
|---|---|
| ≦35 | stress-independent |
| ≧45 | stress-dependent |

The success of simulating conditions in vivo can also be assessed by comparing fluid velocities above the biofilm. Table 3 compares data for both our system and BioFlux™ microfluidics with values estimated for unstimulated saliva film flow over the inner surfaces of three oral regions (Dawes et al., 1989, J. Dent. Res. 68: 1479). While the fluid velocity in an embodiment of our system (i.e. Ibidi flow cell at 600 μL h⁻¹) is within the range of the highest unstimulated flow in vivo (on lingual side of teeth), the fluid velocities calculated from published data on the BioFlux™ system are out of this range.

TABLE 3
Fluid velocities above the biofilm (mm · min⁻¹).

| | |
|---|---|
| Oral lower anterior buccal region | 1.0 |
| Oral upper anterior buccal region | 0.8 |
| Oral lower anterior lingual region | 7.6 |
| Ibidi flow cell at 600 μL h⁻¹ | 6.6 |
| BioFlux attached biofilm growing | 12.0 |
| BioFlux biofilm seeding | 59.8 |

While the exemplary embodiments have been described herein, it is to be understood that the invention is not limited to the disclosed embodiments. The invention is intended to cover various modifications and equivalent arrangements included within the spirit and scope of the appended claims, and scope of the claims is to be accorded an interpretation that encompasses all such modifications and equivalent.

What is claimed is:

1. A continuous flow system for passing fluid over a biofilm to simulate an oral environment, the continuous flow system comprising:
a flow cell housing comprising:
a base defining a longitudinal axis; and
a plurality of channels defined by a plurality of channel walls supported by the base, the plurality of channels distributed adjacent to one another along the longitudinal axis of the base, each channel of the plurality of channels extending transverse to the longitudinal axis of the base, each channel of the plurality of channels having an inflow connection location for receiving the fluid into the channel and an outflow connection location for exporting the fluid from the channel;
a plurality of removable channel connectors, each channel connector defining a connecting channel fluidly coupling a pair of the plurality of channels by connecting the outflow connection location of an upstream channel of the plurality of channels and the inflow connection location of a downstream channel of the plurality of channels;
an upstream inflow adaptor fluidly connected to the flow cell housing for removably connecting to an inflow conduit defining an inflow channel; and
a downstream outflow adaptor connected to the flow cell housing for removably connecting to an outflow conduit defining an outflow channel;
wherein at least one of the plurality of channel walls is for supporting growth of the biofilm, the plurality of channels is for receiving the fluid via the inflow conduit from a reservoir positioned upstream of the flow cell housing, and the outflow channel is for receiving the fluid from the plurality of channels.

2. The continuous flow system of claim 1, wherein a first surface material of the channel connector includes a material that is different than a second surface material of the plurality of channel walls such that the first surface material facilitates less biofilm growth per unit area per unit time relative to the second surface material.

3. The continuous flow system of claim 1, wherein the reservoir is a syringe.

4. The continuous flow system of claim 3, wherein the fluid contains cells for forming the biofilm along a surface of a first channel of the plurality of channels.

5. The continuous flow system of claim 4, wherein the cells are a mixed inoculum.

6. The continuous flow system of claim 1, wherein the height and width of at least one channel of the plurality of channels is greater than 100 μm and greater than 400 μm, respectively.

7. The continuous flow system of claim 1, wherein the height and width of at least one channel of the plurality of channels is about 400 μm and about 3.8 mm, respectively.

8. The continuous flow system of claim 1, wherein an adaptor is connected between the channel connector and the inflow connection location, or between the channel connector and the outflow connection location.

9. The continuous flow system of claim 1, wherein at least one of the plurality of channel connectors defines a bend.

10. The continuous flow system of claim 1, wherein the second surface material facilitates adhesion of cells of the biofilm to the plurality of channel walls.

11. The continuous flow system of claim 2, wherein the second surface material is selected from the group consisting of: collagen I, collagen IV, fibronectin, poly-L-lysine and poly-D-lysine.

12. The continuous flow system of claim 1, wherein at least one of the plurality of channel connectors is removable from the continuous flow system.

13. The continuous flow system of claim 1, wherein at least a portion of the connecting channel has a cross-sectional area that is greater than the cross-sectional area of a channel of the plurality of channels.

14. The continuous flow system of claim 1, wherein the connecting channel is oriented on a different plane than the plurality of channels.

15. The continuous flow system of claim 1 further comprising a hydraulic pump for pumping the fluid from the reservoir to the inflow channel.

16. The continuous flow system of claim 15, wherein the hydraulic pump is a linear pump and the fluid contains a molecular probe for contacting cells of the biofilm.

17. The continuous flow system of claim 1, wherein the plurality of channel walls are integral with the base.

18. The continuous flow system of claim 1, wherein the base has the dimensions of a standard microscope slide.

19. The continuous flow system of claim 18, wherein the base is removably mountable to a microscope stage.

20. The continuous flow system of claim 1, wherein at least one channel of the plurality of channels is directly fluidly coupled to two other channels of the plurality of channels.

21. A method of passing fluid over a biofilm to simulate an oral environment within a flow cell having a plurality of channels defined by a plurality of channel walls supported by a base defining a longitudinal axis, the plurality of channels distributed adjacent to one another along the longitudinal axis of the base, each channel of the plurality of channels extending transverse to the longitudinal axis of the base, the method comprising:
fluidly coupling an inflow channel defined by an inflow conduit to a reservoir containing fluid;
fluidly coupling the inflow channel to a first channel of the plurality of channels;
fluidly coupling the first channel of the plurality of channels to a second channel of the plurality of channels using a channel connector, the channel connector fluidly coupling the first channel to the second channel via a connecting channel defined by a wall of the channel connector;
fluidly coupling an outflow channel defined by an outflow conduit to the second channel of the plurality of channels; and
passing the fluid from the reservoir to the inflow channel such that the fluid flows from the inflow channel to the first channel, from the first channel to the second channel, and from the second channel to the outflow channel to promote growth of the biofilm.

22. The method of claim 21, further comprising the step of inoculating cells for forming the biofilm into the first channel of the plurality of channels prior to said fluidly coupling the first channel of the plurality of channels to the second channel of the plurality of channels.

23. The method of claim 22, wherein the fluid comprises nutrient medium, and said passing the fluid from the first channel of the plurality of channels to the second channel of the plurality of channels promotes distribution of the cells from the biofilm in the first channel of the plurality of channels to the second channel of the plurality of channels.

24. The method of claim 22, wherein the cells are a mixed inoculum.

25. The method of claim 21, wherein a first surface material of the channel connector includes a material that is different than a second surface material of the plurality of channel walls such that the first surface material facilitates less biofilm growth per unit area per unit time relative to the second surface material.

26. The method of claim 21, wherein passing the fluid from the reservoir to the inflow channel further involves passing the fluid through a flow interrupter for inhibiting backflow of the fluid into the reservoir.

27. The method of claim 21, further comprising the step of regulating a flow rate of the fluid flowing from the reservoir.

28. The method of claim 21, wherein the flow cell is supported by a base, and the method further comprises removably mounting the base to a microscope stage.

29. A continuous flow system for passing fluid over a biofilm to simulate an oral environment, the continuous flow system comprising:
a flow cell housing comprising:
a base defining a longitudinal axis; and
a plurality of channels defined by a plurality of channel walls supported by the base, the plurality of channels distributed adjacent to one another along the longitudinal axis of the base, each channel of the plurality of channels extending transverse to the longitudinal axis of the base, each channel of the plurality of channels having an inflow connection location for receiving the fluid into the channel and an outflow connection location for exporting the fluid from the channel;
an upstream inflow adaptor connected to the flow cell housing for removably connecting to an inflow conduit defining an inflow channel for directing the fluid to the plurality of channels;
a downstream outflow adaptor connected to the flow cell housing for removably connecting to an outflow conduit defining an outflow channel for receiving the fluid from the plurality of channels; and
a removable reservoir fluidly connected to the plurality of channels via the inflow conduit for supplying the fluid to the plurality of channels;
wherein at least one of the plurality of channel walls is for supporting growth of the biofilm, the plurality of channels receives the fluid from the reservoir via the inflow conduit, and the outflow channel receives the fluid from the plurality of channels.

30. The continuous flow system of claim 29, wherein the plurality of channels receives the fluid from the reservoir via a flow interrupter positioned downstream of the reservoir and upstream of the plurality of channels for inhibiting backflow of the fluid into the reservoir.

31. The continuous flow system of claim 30, wherein a glass shield is mounted adjacent the reservoir to inhibit contamination of the fluid in the reservoir by covering an opening of the reservoir.

32. The continuous flow system of claim 30, further comprising a second reservoir positioned downstream of the flow interrupter and upstream of the plurality of channels for supplying the fluid to the plurality of channels.

33. The continuous flow system of claim 32, wherein flow of the fluid from the reservoir into the plurality of channels and from the second reservoir into the plurality of channels is regulated by a 3-way stopcock.

34. The continuous flow system of claim 32, wherein the second reservoir is a syringe.

35. A continuous flow system for passing fluid over a biofilm to simulate an oral environment, the continuous flow system comprising:
a flow cell housing comprising:
a base defining a longitudinal axis; and
a plurality of channels defined by a plurality of channel walls supported by the base, the plurality of channels distributed adjacent to one another along the longitudinal axis of the base, each channel of the plurality of channels extending transverse to the longitudinal axis of the base, each channel of the plurality of channels having an inflow connection location for receiving the fluid into the channel and an outflow connection location for exporting the fluid from the channel;
the fluid passed over the biofilm during a first stage of operation to produce a first stage shear stress at a first pre-determined shear stress range, a first stage fluid velocity at a first pre-determined fluid velocity range, and a first stage dilution rate at a first pre-determined dilution rate range;
the fluid passed over the biofilm during a second stage of operation to produce a second stage shear stress at a second pre-determined shear stress range, a second stage fluid velocity at a second pre-determined fluid velocity range, and a second stage dilution rate at a second pre-determined dilution rate range, at least one of the second pre-determined shear stress range, the second pre-determined fluid velocity range and the second pre-determined dilution rate range being outside of the respective corresponding first pre-determined shear stress range, first pre-determined fluid velocity range, and first pre-determined dilution rate range.

36. The continuous flow system of claim 35, further comprising a plurality of removable channel connectors, each channel connector defining a connecting channel fluidly coupling a pair of the plurality of channels by connecting the outflow connection location of an upstream channel of the plurality of channels and the inflow connection location of a downstream channel of the plurality of channels.
